(19) United States
(12) Patent Application Publication
(10) Pub. No.: US 2019/0192502 A1
(43) Pub. Date: Jun. 27, 2019

(54) Modulation of Transcription Initiation Factor TFIID Subunit 1 (TAF1) for Treating Leukemia

(71) Applicant: UNIVERSITY OF MIAMI, Miami, FL (US)

(72) Inventors: Stephen Nimer, Miami, FL (US); Ye Xu, Miami, FL (US)

(21) Appl. No.: 16/191,213

(22) Filed: Nov. 14, 2018

Related U.S. Application Data
(60) Provisional application No. 62/586,550, filed on Nov. 15, 2017.

(51) Int. Cl. A61K 31/473; A61P 35/02

(52) U.S. Cl. CPC A61K 31/473; A61P 35/02

(57) ABSTRACT

The disclosure provides a method of treating leukemia, the method comprising administering to mammalian subject in need thereof an inhibitor of Transcription initiation factor TFIID subunit 1 (TAF1). The disclosure further provides a method of reducing the risk of leukemia, the method comprising administering to mammalian subject in need thereof an inhibitor of Transcription initiation factor TFIID subunit 1 (TAF1).

### CROSS-REFERENCE TO RELATED APPLICATIONS

This application claims priority to U.S. Provisional Patent Application No. 62/586,550, filed on Nov. 15, 2017, the disclosure of which is hereby incorporated by reference in its entirety.

### GRANT FUNDING DISCLOSURE

This invention was made with government support NIH-NCI grant number RO1CA166835. The government has certain rights in the invention.

### FIELD OF THE INVENTION

The disclosure relates to methods of modulating transcription initiation factor TFIID subunit 1 (TAF1) in the treatment of leukemia.

### BACKGROUND

The t(8;21)(q22;q22) is the most commonly observed chromosomal translocation in Acute Myelogenous Leukemia (AML) patients. It generates the AML1-ETO (AE) fusion protein¹⁻⁴, which contains the N-terminal 177 amino acids of Acute Myelogenous Leukemia 1 [AML1, also known as RUNX1 (runt-related transcription factor 1)] fused to nearly the entire Eight Twenty One (ETO) protein¹⁻⁴. Both RUNX1 and its non-DNA binding partner protein CBFβ (Core Binding Factor beta subunit) are essential for definitive hematopoiesis during embryogenesis^{5, 6}. AE impairs myeloid differentiation and promotes the self-renewal of hematopoietic stem cells⁷⁻⁹, both of which are critical for AE driven leukemia development. The importance of AE in leukemia development makes it an attractive therapeutic target¹⁰⁻¹³, yet targeting it directly has been difficult¹⁴.

### SUMMARY

The disclosure is directed to methods of treating and/or reducing the risk of leukemia, the method comprising administering to mammalian subject in need thereof an inhibitor of Transcription initiation factor TFIID subunit 1 (TAF1). In various aspects, the mammalian subject is a human. In certain aspects the inhibitor of TAF1 is a TAF1 bromodomain inhibitor.

In certain aspects, the leukemia is Acute Myelogenous Leukemia (AML). In certain aspects, the leukemia is Acute Myelogenous Leukemia 1-Eight Twenty One oncoprotein (AML1-ETO) expressing leukemia.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. In addition, the invention includes, as an additional aspect, all embodiments of the invention narrower in scope in any way than the variations specifically mentioned above. With respect to aspects of the invention described or claimed with “a” or “an,” it should be understood that these terms mean “one or more” unless context unambiguously requires a more restricted meaning. With respect to elements described as one or more within a set, it should be understood that all combinations within the set are contemplated. If aspects of the invention are described as “comprising” a feature, embodiments also are contemplated “consisting of” or “consisting essentially of” the feature. Additional features and variations of the disclosure will be apparent to those skilled in the art from the entirety of this application, and all such features are intended as aspects of the disclosure.

### BRIEF DESCRIPTION OF THE DRAWINGS

FIGS. 1A-1F. The lack of TAF1 blocked proliferation and induced apoptosis in AE expressing cells. (FIG. 1A) Knockdown of TAF1 blocked the growth of Kasumi-1 cells. (FIG. 1B) Knockdown of TAF1 impairs the growth of SKNO-1 cells. (FIG. 1C) Depletion of TAF1 had little influence on the growth of K562 cells. (FIG. 1D) TAF1 is dispensable for the growth of CD34+ cells. Kasumi-1 cells, SKNO-1 cells, K562 cells and CD34+ cells were infected with scrambled shRNA or TAF1 directed shRNAs. The levels of TAF1 depletion by two different TAF1 directed shRNAs in each type of cells are shown on the right. (FIG. 1E) Knockdown of TAF1 reduced the percentage of Kasumi-1 cells in S phase and had no influence in K562 and CD34+ cells. (FIG. 1F) Lack of TAF1 induced apoptosis in AE expressing cells. Kasumi-1 cells, K562 cells and CD34+ cells were transduced with scrambled shRNA or hTAF1 shRNA for 4 days and then subjected for Brdu assay or apoptotic assays.

FIGS. 2A-2E. TAF1 deficiency promoted myeloid differentiation and blocked self-renewal of hematopoietic stem cells. (FIGS. 2A-2B) Knockdown of TAF1 reversed the blockade of myeloid differentiation driven by AE in CD34+ cells (FIG. 2A) and bone marrow cells (FIG. 2B). To monitor myeloid differentiation, CD34+ cells were incubated in myeloid differentiation promoting medium for 4 days. Mac1 was used as the myeloid differentiation marker to stain CD34+ cells. The myeloid differentiation markers mac1 and Gr1 were used to stain bone marrow cells. (FIG. 2C) Knockdown of TAF1 reduced the self-renewal induced by AE. Serial plating assays were performed using bone marrow cells isolated from Mx1-cre or AE knockin mice and transduced with scrambled shRNA or TAF1 directed shRNA. The numbers of colonies at each plating are shown as average±SD, n=3. (FIGS. 2D-2E) Knockdown of TAF1 reversed cobblestone area forming cell (CAFC) frequency induced by AE in bone marrow cells (FIG. 2D) and CD34+ cells (FIG. 2E). Bone marrow cells were isolated from Mx1-cre or AE knockin mice and transduced with scrambled shRNA or mTAF1 shRNA. CD34+ cells were infected with scrambled shRNA or hTAF1 shRNA for 2 days. After puromycin selection, CD34+ cells were transduced with either migr1 or migr1-AE viruses for 3 days and GFP+ migr1 or migr1-AE expressing CD34+ cells were sorted and plated for CAFC assays. The numbers of cobblestone area were counted at week 5 and shown as average±SD, n=3. * indicates p<0.05, *** indicates p<0.001. NS indicates p>0.05. p values were determined by Student t test.

FIGS. 3A-3E. Depletion of TAF1 affected proliferation and self-renewal of AE9a expressing cells. (FIG. 3A) Schema of the generation of AE9a luciferase cells and the collection of secondary spleen leukemic cells. (FIG. 3B) Knockdown of TAF1 significantly reduced the proliferation of secondary spleen leukemic cells. Secondary spleen leukemic cells were transduced with scrambled shRNA and mouse TAF1 directed shRNAs. All groups started with the same cell numbers, cell numbers were counted on days 3, 5 and 7. (FIG. 3C) TAF1 is critical for the self-renewal of secondary spleen leukemic cells. Serial plating assays were performed using secondary spleen leukemic cells transduced with scrambled shRNA, mouse TAF1 directed shRNAs. (FIG. 3D) TAF1 is important for maintaining CAFC frequency in secondary spleen leukemic cells. CAFC assays were performed using secondary spleen leukemic cells transduced with scrambled shRNA and mouse TAF1 directed shRNAs. The numbers of cobblestone area were counted at week 5 and shown as average±SD, n=3. ** indicates p<0.01, p values were determined by Student t test. (FIG. 3E) Depletion of TAF1 impaired the expression of AE9a target genes in AE9a cells. AE9a cells were transduced with scrambled shRNA or mTAF1 shRNAs for 5 days and the mRNA levels of individual genes were standardized by mouse 18S rRNA level.

FIGS. 4A-4F. The knockdown of TAF1 significantly delayed leukemia development. (FIG. 4A) Knockdown of TAF1 significantly extended the survival of recipient mice transplanted with AE9a luciferase cells. CB57Bl/6J mice were irradiated at 450 cGys and injected with AE9a luciferase cells transduced with scrambled shRNA or mouse TAF1 shRNAs. (n=8 in each group) (FIG. 4B) KD of TAF1 reduced the growth of GFP+AE9a luciferase cells in the peripheral blood. Mice were injected with GFP+AE9a luciferase cells infected with scrambled shRNA or TAF1 shRNAs. The percentage of GFP+AE9a luciferase cells in peripheral blood of each mouse was measured 3 weeks after the transplantation. (FIG. 4C) In vivo luciferase imaging indicated that the depletion of TAF1 remarkably impairs leukemia development. (n=8 in each group.) Mice were injected with AE9a luciferase cells expressing wildtype level or reduced levels of TAF1. 20 days after transplantation, IVIS imaging was performed. (FIG. 4D) The quantification of total luciferase signal in each mouse of each group as shown in (FIG. 4C). (FIG. 4E) Survival curves of mice injected with secondary spleen leukemic cells transduced with scrambled shRNA or TAF1 directed shRNAs. n=8 mice in each group. (FIG. 4F) The percentage of GFP+AE9a cells in the peripheral blood of each mouse after receiving secondary spleen leukemic cells infected with scrambled shRNA or TAF1 directed shRNAs. Peripheral blood was collected 48 days after transplantation. * indicates p<0.05, ** indicates p<0.01, **** indicates p<0.0001. p values were determined by Student t test.

FIGS. 5A-5E. TAF1 associates with acetylated K43 on AE through its bromodomains. (FIG. 5A) TAF1 physically interacts with AE in Kasumi-1 cells. Co-immunoprecipitation was performed using anti-TAF1 antibody or normal mouse IgG. (FIG. 5B) Co-immunoprecipitation of TAF1 with AE using anti-ETO antibody or normal goat IgG. (FIG. 5C) Lysine 43 mutation on AE blocked the interaction of TAF1 with AE. 293T cells were transfected with p300 and AE or its mutants and TAF1. Co-immunoprecipitation was performed using anti-TAF1 antibody. (FIG. 5D) The deletion of the TAF1 bromodomain regions impaired its binding to AE. 293T cells were transfected with p300, AE and TAF1 wildtype or bromodomain deletion (ΔBr) plasmids. Co-immunoprecipitation was performed using anti-ETO antibody. (FIG. 5E) Mass spectrometry analysis of protein associated with TAF1. Co-immunoprecipitation was performed using an anti-TAF1 antibody. “Unique” indicates the number of unique peptide matches to the protein; “total” indicates the total number of peptides matched to the protein. “AVG” indicates the average Xcorr value for all the peptides matched to the protein. Xcorr is a score used by Sequest (the search algorithm) to judge the quality of the spectral match to a particular peptide sequence.

FIGS. 6A-6E. The depletion of TAF1 blocks the binding of AE to chromatin and thereby represses the expression of AE target genes. (FIG. 6A) The influence of AE knockdown on mRNA levels of AE target genes in Kasumi-1 cells. Kasumi-1 cells were transduced with scrambled shRNA or AE shRNAs for 4 days. mRNA levels of individual genes were standardized by 18S rRNA level. (FIG. 6B) The impact of TAF1 knockdown on mRNA levels of AE target genes in Kasumi-1 cells. Kasumi-1 cells were transduced with scrambled shRNA or hTAF1 shRNAs for 4 days. The mRNA levels of individual genes were standardized by 18S rRNA level. (FIG. 6C) The protein levels of AE target genes in Kasumi-1 cells and K562 cells infected with scrambled shRNA or hTAF1 shRNA. (FIG. 6D) The knockdown of TAF1 reduces the recruitment of AE to regulatory regions of ID1 gene. Kasumi-1 cells expressing wildtype levels or reduced levels of TAF1 were subjected to ChIP assay. Primers against AE binding site or non-AE binding site were used for realtime PCR. The AE binding site is 670 bp from the TSS of the ID1 gene and the non AE binding site is 3 kb from the TSS of the ID1 gene. (FIG. 6E) Knockdown of TAF1 reduced the amount of AE in the Kasumi-1 chromatin fraction. Kasumi-1 cells were transduced with scrambled shRNA or hTAF1 shRNA for 3 days and then collected for a subcellular fractionation assay. “Total” indicates the whole cell lysate; “cyto” indicates cytoplasm fraction; “mem” indicates membrane bound fraction; “NS” indicates nuclear soluble fraction; “chrom” indicates chromatin fraction.

FIGS. 7A-7D. TAF1 cooperates with AE to control the expression of both AE upregulated and downregulated genes. (FIG. 7A) Venn diagrams of all genes differentially upregulated or downregulated (q<0.05) in Kasumi-1 cells after TAF1 KD (shTAF1) or AE KD (shAE). Scrambled shRNA infected cells were used as controls. Enrichment analysis for gene ontology (GO) biological processes and Kyoto Encyclopedia of Genes and Genomes (KEGG) gene sets was performed on overlapping genes significantly differentially expressed in both shAE and shTAF1 conditions. The red line indicates a significance threshold of q<0.05. (FIG. 7B) Venn diagram illustrating the number of overlapping called peaks (q<0.05) in anti-TAF1 antibody and anti-ETO antibody ChIP-seq samples in Kasumi-1 cells. (FIG. 7C) ChIP-seq analysis shows the TAF1 or IgG signal (in read count per million) at the TSS of AE activated genes and repressed genes. (FIG. 7D) Venn Diagram illustrating the number of overlapping peaks in anti-TAF1 antibody and anti-ETO antibody ChIP-seq samples at the TSS (within 1 kb of the transcription start-sites) of all differentially expressed genes or TAF1 upregulated or downregulated genes after TAF1 KD (q<0.05).

FIGS. 8A-8F. The effect of TAF1 bromodomain inhibitor Bay-364 on the growth of Kasumi-1 cells, K562 cells and CD34+ cells. (FIGS. 8A-8C) The growth of Kasumi-1 cells, CD34+ cells and K562 cells in the presence or absence of different concentrations of bromodomain inhibitors Bay-364, Bay-299 or JQ-1. The cell growth was measured by CellTiter-Glow luminescent cell viability assay after 3 days of treatment with the inhibitors. Each treatment was in triplicate and shown as average±SD. (FIG. 8D) IC50s (μM) of inhibitors on Kasumi-1, CD34+ and K562 cellsKasumi-1 cells, CD34+ cells and K562 cells were treated with different concentrations of Bay-364, Bay-299 and JQ-1 for three days and cell growth was measured by CellTiter-Glow luminescent cell viability assay. Each treatment was in triplicate and IC50s were calculated using GraphPad Prism. (FIG. 8E) TAF1 inhibitor Bay-364 repressed the expression of AE upregulated genes. RNA was extracted from Kasumi-1 cells treated with or without Bay-364 for 72 hours. mRNA levels of individual genes were standardized by 18S rRNA level. (FIG. 8F) TAF1 working model: Under normal conditions, the acetylation of K43 on AE by p300 is recognized by the bromodomains of TAF1 and TAF1 enhances the binding of AE to chromatin. Then transcription is activated. In absence of TAF1, the binding affinity of AE with chromatin is abrogated, and transcription is blocked.

FIGS. 9A-9E. The knockdown of TAF1 repressed AE promoting self-renewal. (FIG. 9A) AE was expressed in AE knockin mice (left panel) and TAF1 was knocked down using mTAF1 shRNA in bone marrow cells isolated from Mx1-cre mice and AE knockin mice (right panel). (FIG. 9B) AE expression was induced by poly(I:C) (left panel) and TAF1 was knocked down using mTAF1 shRNA #2 in bone marrow cells collected from Mx1-cre mice and AE knockin mice (right panel). (FIG. 9C) Lack of TAF1 blocks self-renewal activated by AE. The serial plating assays were performed using bone marrow cells collected from Mx1-cre or AE knockin mice and transduced with scrambled shRNA or mTAF1 shRNA#2. (FIG. 9D) TAF1 depletion levels in CD34+ cells. CD34+ cells were transduced with scrambled shRNA or TAF1 direct shRNA for 2 days and then subjected for puromycin selection for additional 2 days. mRNA was extracted from selected cells. (FIG. 9E) TAF1 depletion levels in secondary spleen leukemic cells. Secondary spleen leukemic cells were infected with scrambled shRNA or TAF1 direct shRNAs for 3 days and mRNA were extracted.

FIGS. 10A-10D. TAF1 is critical for leukemia development. (FIG. 10A) The loss of TAF1 did not affect the homing of AE9a luciferase cells to bone marrow. Bone marrow cells were collected 16 hours after mice were injected with AE9a luciferase cells transduced with scrambled shRNA or mTAF1 shRNA#1. p=0.62 (FIG. 10B) The mRNA level of TAF1 in AE9a luciferase cells after transduction of scrambled shRNA or mTAF1 shRNAs. (FIG. 10C) The mRNA level of TAF1 in secondary spleen leukemic cells transduced with scrambled shRNA or mTAF1 shRNAs. (FIG. 10D) The lack of TAF1 abrogated the infiltration of leukemic cells in peripheral blood. Representative images show the HE staining of peripheral blood from mice transplanted with secondary spleen leukemic cells infected with scrambled shRNA or mTAF1 shRNs for 7 weeks.

FIG. 11. The knockdown of TAF1 decreased the amount of AE in chromatin fraction. Kasumi-1 cells were transduced with scrambled shRNA or TAF1 shRNA#2 for 5 days and then collected for a subcellular fractionation assay. “cyto” indicates cytoplasm fraction; “NS” indicates nuclear soluble fraction; “chrom” indicates chromatin fraction; “NIS” indicates nuclear insoluble fraction.

FIGS. 12A-12E. The knockdown of TAF1 reduced the expression of a subset of AE regulated genes. (FIGS. 12A-12B) Heat maps of differentially expressed genes in Kasumi-1 cells infected with AE shRNAs (FIG. 12A) or TAF1 shRNAs (FIG. 12B) compared to those infected with scramble shRNAs. Heat maps were generated from variance stabilized counts of genes differentially expressed with a BH FDR q<0.05 and a fold change+/−1.5 vs control. (FIGS. 12C-12D) Scatter plot of average variance stabilized log transformed counts of genes changing with AE KD (FIG. 12C) or TAF1 KD (FIG. 12D), compared to scramble shRNA controls. Significantly up- and down-regulated genes with a fold change cutoff of 1.5 are highlighted as indicated. (FIG. 12E) Non-normalized signal read count density tracks of indicated samples at ID1 and CARM1 genes. Estimated library sizes of displayed samples are within 5%.

FIGS. 13A-13C. TAF1 co-occupies with AE at a subset of AE target genes. (FIG. 13A) AE, not TAF1 is recruited at SPI1 promoter region. Kasumi-1 cells were infected with scrambled shRNA or hTAF1 shRNA. Chromatin immunoprecipitation was performed using anti-TAF1 antibody and anti-ETO antibody. Real time PCR was performed using primers against promoter region and non-promoter region of SPI1 gene. (FIG. 13B) TAF1 co-localizes with AE at ID1 gene. ChIP-seq was performed using anti-TAF1 and anti-ETO antibodies in Kasumi-1 cells. Scale in signal per million reads. (FIG. 13C) Heatmaps of TAF1 and ETO read density centered on the transcription start-site (TSS) of all differentially expressed genes after TAF1 KD (q<0.05). No cutoff was used.

### DETAILED DESCRIPTION

The disclosure relates to methods of treating or reducing the risk of leukemia, the method comprising administering to mammalian subject in need thereof an inhibitor of Transcription initiation factor TFIID subunit 1 (TAF1). In certain aspects the disclosure provides methods for treating or reducing the risk of Acute Myelogenous Leukemia (AML) using a bromodomain inhibitor.

### Transcription Initiation Factor TFIID Subunit 1 (TAF1)

Transcription is a highly regulated multiple-step process in eukaryotes starting with the assembly of a preinitiation complex (PIC). For RNA polymerase II dependent transcription, PIC assembly involves the loading of activators at enhancers, the binding of TATA-binding protein (TBP) to TATA-containing promoters and the subsequent recruitment of TAF1 (also termed TAFII250), the largest subunit of the transcription factor IID complex (TFIID). TAF1 serves as a bridge to bring 12 more TAFs to promoter regions¹⁵. Recently, the various modes of assembly of the preinitiation complex and combinations of TFIID components have been described as promoter-specific, tissue-specific or cell type-specific¹⁶⁻¹⁹. For instance, TAF1 is absent from human embryonic stem cells and its overexpression in those cells triggers their differentiation¹⁶.

In various aspects, the method comprises administering to a subject in need thereof an inhibitor of Transcription initiation factor TFIID subunit 1 (TAF1), such as a TAF1 bromodomain inhibitor. By “TAF1 bromodomain inhibitor” is meant a bromodomain inhibitor that reduces or inhibits the activity of TAF1. Suitable TAF1 bromodomain inhibitor include, but are not limited to, Bay-364 (6-(3-Hydroxy-propyl)-2-(1-methyl-2-oxo-2,3-dihydro-1H-benzoimidazol-5-yl)-benzo[de]isoquinoline); Bay-299 (6-(3-Hydroxypropyl)-2-(1,3,6-trimethyl-2-oxo-2,3-dihydro-1Hbenzimidazol-5-yl)-1H-benzo[de]isoquinoline-1,3(2H)-dione), 2-(1,3,6-Trimethyl-2-oxo-2,3-dihydro-1H-benzimidazol-5-yl) 1Hbenzo[de]isoquinoline-1,3(2H)-dione; 2-[6-(Dimethylamino)-1,3-dimethyl-2-oxo-2,3-dihydro-1H-benzimidazol-5-yl]-1H-benzo[de]isoquinoline-1,3(2H)-dione; 2-(6-Bromo-1,3-dimethyl-2-oxo-2,3-dihydro-1H-benzimidazol-5-yl)-1H-benzo[de]isoquinoline-1,3(2H)-dione; 6-Bromo-2-(1,3,6-trimethyl-2-oxo-2,3-dihydro-1H-benzimidazol5-yl)-1H-benzo[de]isoquinoline-1,3(2H)-dione; 6-Chloro-2-(1,3,6-trimethyl-2-oxo-2,3-dihydro-1H-benzimidazol5-yl)-1H-benzo[de]isoquinoline-1,3(2H)-dione; 5-Nitro-2-(1,3,6-trimethyl-2-oxo-2,3-dihydro-1H-benzimidazol-5-yl)-1H-benzo[de]isoquinoline-1,3(2H)-dione; 5-Amino-2-(1,3,6-trimethyl-2-oxo-2,3-dihydro-1H-benzimidazol5-yl)-1H-benzo[de]isoquinolone-1,3(2H)-dione; 5-Hydroxy-2-(1,3,6-trimethyl-2-oxo-2,3-dihydro-1H-benzimidazol-5-yl)-1H-benzo[de]isoquinoline-1,3(2H)-dione; 5-Bromo-2-(1,3,6-trimethyl-2-oxo-2,3-dihydro-1H-benzimidazol5-yl)-1H-benzo[de]isoquinoline-1,3(2H)-dione; or 1,3-Dioxo-2-(1,3,6-trimethyl-2-oxo-2,3-dihydro-1H-benzimidazol-5-yl)-2,3-dihydro-1H-benzo[de]isoquinoline-5-carbonitrile.

### Acute Myelogenous Leukemia 1-Eight Twenty One Oncoprotein (AML1-ETO)

AML1-ETO (AE) is a fusion transcription factor, generated by t(8;21), that functions as a leukemia promoting oncogene. Herein, it is demonstrated that the bromodomains of TAF1 associate with K43 acetylated AE, and this association plays a pivotal role in the proliferation of AE expressing AML cells. Depletion of TAF1 impaired the recruitment of AE to its target genes, interfering with its control on the expression of both AE upregulated and downregulated genes. As described herein, TAF1 is essential for AE driven leukemogenesis. Together, these findings reveal a novel role of TAF1 in leukemogenesis and identify TAF1 as an alternative therapeutic target for AE expressing leukemia.

### Experimental Procedure

Plasmid construction: The pLKO.1 plasmid expressing two human TAF1 shRNAs (hTAF shRNA#1 and hTAF1 shRNA#2) and two mouse TAF1 shRNAs (mTAF1 shRNA#1 and mTAF1 shRNA#2) were purchased from Sigma. Migr1, Migr1-AE and its amino acid mutation plasmids were described in Wang et al. 2011. TAF1 cDNA was purchased from Addgene, the full length sequence was corrected and verified by DNA sequencing and reconstructed into pCDH-MSCV-EF1 vector purchased from SBI Biotech. TAF1 bromodomain deletion (ABr) construct was cloned by deleting amino acids 1397-1510, using PCR based mutagenesis.

Cord Blood CD34+ Cell Purification, Colony Forming Assays, Cobblestone Area Forming Cell (CAFC) Assays and Liquid Culture Differentiation Assays:

Human cord blood was purchased from the New York Blood Center. The purification of CD34+ cells was described previously 25. Purified CD34+ cells were transduced with scrambled shRNA or TAF1 directed shRNA followed by puromycin selection for 48 hours. After puromycin selection, GFP tagged Migr1 or Migr1-AE retroviruses were introduced into CD34+ cells and the GFP+CD34+ cells were sorted using FACS Aria IIu (BD Biosciences). CD34+ cells expressing either Migr1 or Migr1-AE were resuspended in Methocult GF M3434 medium (Stem Cell Technologies) and replated into 6 well plate with a density of 3000 cells per well for colony assay. Seven days after the initial plating, colonies were counted and then all the cells were collected for replating weekly with a density of 3000 cells per well for continuous 4 weeks. Cobblestone area forming cell assays (CAFC) were performed as described previously 25, the numbers of total cobblestone area were counted after 5 weeks. To monitor myeloid differentiation, sorted GFP+CD34+ cells were grown in myeloid differentiation medium X-VIVO supplemented with 20% BIT 9500 (Stem Cell Technologies), SCF 100 ng/ml, Flt3-L 10 ng/ml, 11-3 20 ng/ml, G-CSF 20 ng/ml, GM-CSF 20 ng/ml and IL-6 20 ng/ml (PeproTech) for 4 days, the expression of myeloid differentiation markers mac1 was measured by flow cytometry.

Bone Marrow Cell Isolation:

Poly(I:C) (10 mg/kg was given every other day for three times) was administered to Mx1-cre and AE knockin mice to induce AE expression. Ten days after induction, tail blood was collected to assess AE expression. Four days before isolating bone marrow stem cells, 5FU (150 mg/kg) was injected into the mice to deplete proliferating blood cells. Two weeks after poly(I:C) application, mice were killed and bone marrow cells were isolated from femurs and tibias. During the preparation of bone marrow cells, ACK (Ammonium-Chloride-Potassium) lysing buffer was used to lyse red blood cells. Isolated bone marrow cells were plated in IMDM medium supplemented with 10% fetal bovine serum and cytokines (SCF 100 ng/ml, IL-6 20 ng/ml and IL-3 10 ng/ml).

Leukemia mouse models: Fetal liver cells were isolated from E14.5 embryos of C57BL/6 mice and infected with GFP tagged Migr1-AE9a retroviruses. GFP positive fetal liver cells were sorted by flow cytometry. 6-8 weeks female C57Bl/6.SJL recipient mice were purchased from The Jackson Laboratory and lethally irradiated with 950 cGys. After irradiation, recipient mice were transplanted with GFP positive fetal liver cells through tail-vein injection. Five to six weeks after transplantation, primary leukemic cells were collected from the bone marrow of mice developed leukemia and injected into second batch of recipient mice for secondary transplantation. After 5-6 weeks, secondary spleen leukemic cells were collected from mice developed leukemia after secondary transplantation. Primary bone marrow cells were also grown in RPMI 1640 with 20% fetal bovine serum for weeks to develop AE9a cells. Luciferase gene was integrated into AE9a cells to create AE9a luciferase cells. AE9a luciferase cells or secondary spleen leukemic cells were transduced with scrambled shRNA or mTAF1 shRNA#1 or mTAF1 shRNA#2. After being confirmed the depletion of TAF1, the secondary spleen leukemic cells or AE9a luciferase cells were injected into C57BL/6 recipient mice which have received sublethal irradiation (450 cGys). Three weeks after transplantation, the percentage of GFP+AE9a luciferase cells in the peripheral blood was monitored by flow cytometry every week. The distribution of luciferase positive AE9a cells in mice body were monitored using IVIS imaging system once a week.

Statistical Analysis:

Significance were calculated using two tailed student t test. Survival curve were analyzed using Kaplan Meier method in GraphPad Prism 6.0 software.

Cell Lines:

Kasumi-1 cells were grown in RPMI 1640 supplemented with 20% fetal bovine serum, SKNO-1 cells were grown in RPMI 1640 with 10% fetal bovine serum and 10 ng/ml GM-CSF. K562 cells were grown in IMDM with 10% fetal bovine serum.

Chemicals:

Bromodomain inhibitors Bay-299N, Bay-299 and JQ-1 were purchased from Sigma.

Lentivirus, Retrovirus Production and Concentration:

Lentiviruses were produced in 293T cells using lipofectamine 2000 as transfection reagent and psPAX2 and VSVG as packaging plasmids. Retroviruses were produced using Calcium Phosphate Transfection Kit from Sigma-Aldrich following manufacturer's instruction. Viruses were collected 48 hours and 72 hours after transfection and concentrated by lenti-X concentrator or retro-X concentrator (Clontech).

BrdU Assay:

BrdU assay was performed using BD Pharmingen BrdU Flow Kit. Briefly, Kasumi-1 cells and CD34+ cells were transduced with scrambled shRNA and hTAF1 shRNAs. 4 days after transduction, cells were incubated with 100 Brdu for 1 hour. After fixation and permeabilization, cells were digested by DNase at 37° C. for 1 hour. Following anti-BrdU staining, BrdU incorporation was analyzed by flow cytometry.

Subcellular Fractionation Assay:

Subcellular fractionation assay of Kasumi-1 cells was performed using Subcellular Protein Fraction Kit for cultured cells (Thermo Scientific) according to Manufacturer's instruction. Briefly, Kasumi-1 cells were transduced with either scrambled or hTAF1 shRNAs for 3 or 5 days. Equal number of cells from each treatment were collected and used for protein fractionation. Cellular proteins were fractionated to cytoplasmic, membrane bound, nuclear soluble, chromatin bound and nuclear insoluble proteins. β-actin, histone H4, Lamin B were used for loading control of each fraction.

Flow Cytometry:

To monitor the expression of cell surface markers c-kit, sca1, mac1 and gr1, cells were stained by APC conjugated c-kit, PE-cy7 conjugated sca1, PE conjugated mad and percp-cy5.5 conjugated gr1 antibodies purchased from BD Biosciences. To monitor apoptosis, cells were stained with PE conjugated annexin V and 7-AAD using PE Annexin V Apoptosis Detection Kit I from BD Biosciences. To evaluate the percentage of cells in S phase, cells were incubated in 10 μM FITC-Brdu solution for 1 hour. After fixation and permeabilization, cells were digested in 300 μg/ml DNase solution at 37° C. for 1 hour and then subjected for 7-AAD staining. Stained cells were evaluated using FACS Canto-II and data were analyzed by FlowJo_V10 software.

Chromatin Immunoprecipitation and ChIP-Sequencing:

Chromatin immunoprecipitation (ChIP) assays were performed using SimpleChIP Enzymatic Chromatin IP kit (Cell Signaling Technology) following manufacturer instructions. In brief, cells were fixed and lyzed in ChIP buffer. After sonication, insoluble debris was removed by centrifugation. 10% of each supernatant was used as input. Remaining supernatant were diluted in ChIP buffer and incubated with antibody overnight at 4° C. Magnetic protein A/G beads precoated with sperm DNA were added for 1 hour before extensive washes. Immunoprecipitated chromatin fragments were digested with proteinase K and the crosslink between DNA and proteins was reversed at 65° C. for 2 h. DNA was isolated by either spin columns or phenol/chloroform extraction and quantitated by RT-PCR or subjected for ChIP-sequencing library preparation.

ChIP-sequencing was performed at Oncogenomic Core Facility at the Sylvester Comprehensive Cancer Center. IP samples and input were sequenced using single-end reads with an Illumina NextSeq 500. Reads were trimmed for adapters using Skewer [v0.2.2]-q 20 -l 18. Fastq files were aligned to human GRCh38.p3 using BWA [v0.7.13] with parameters aln -q 5 -l 32 -k 2. Peaks were called using macs2 [v2.1.1.20160309] with parameters -SPMR -nomodel -qvalue 0.05 -shift 80 -extsize 180 for AE and 160 for TAF1. Shift and extension sizes were determined using phantompeakqualtools [v1.1]. Ngsplot [v2.61] was used to generate TAF1 and AE binding heatmaps. ChIPseeker [v1.10.0] was used for peak annotation. Bedtools [v2.26.0] intersect was used to determine peak overlaps.

RNA Isolation, Quantitative PCR and RNA-Sequencing:

RNA was extracted using RNeasy mini kit (Qiagen) and cDNA was generated by QuantiTect Rev. Transcription Kit from Qiagen following manufacturer's instructions. The thermal cycle conditions to amplify cDNA were 48° C. for 15 min; 95° C. for 10 min, followed by 40 cycles of 95° C. for 15 s; 60° C. for 1 min and GAPDH or 18S was used as internal control.

In total, 6 samples were collected from TAF1 knockdown cells, comprised of two distinct shRNAs and two independent experiments, and 5 samples were collected from cells infected with scrambled shRNA; 4 samples were collected from either AE knockdown Kasumi-1 cells or cells infected with scrambled shRNA, comprised of two distinct shRNAs. RNA was extracted using RNeasy plus micro kit (Qiagen). Library preparation and RNA-sequencing were done at Oncogenomic Core Facility at the Sylvester Comprehensive Cancer Center. Samples were sequenced using paired ends with an Illumina NextSeq 500 and subsequent sequencing reads were trimmed and filtered using Skewer [v0.2.2]-q 20 -l 18. Fastq files were aligned to Ensembl 87: GRCh38.p7 human transcriptome using STAR aligner [v2.5.3a] and RSEM [v1.3.0] to obtain expected gene counts. Differential expression was determined between TAF1 shRNA or AE shRNA and scrambled shRNA using DESeq2 [v1.14.1] and R [v3.3.1] with a Benjamini-hochberg FDR cutoff of 0.05. Heatmaps were generated using euclidean distances between sample blind, variance stabilized transformed counts from DESeq2. Example gene signal tracks were generated in IGV [v.3.88] using reads files with comparable library sequencing depth (estimated library scaling within 5%). Enrichment analysis of differentially expressed genes in AE and TAF1 experiments was performed using EnrichR [2016 update].

Co-Immunoprecipitation, Western Blot and Antibodies:

Co-immunoprecipitation (co-IP) was performed in NETN buffer as described previously (Stewart et al., 2003). In brief, cell pellet was lysed in NETN buffer (50 mM Tris pH=7.5; 150 mM NaCl; 1 mMEDTA; 1% NP40, phosphatase inhibitor and protease inhibitor cocktail purchased from Roche) with sonication and then incubated at 4° C. for 1 hour. Insolvable debris was removed by centrifugation. Following preclearing, an equal amount of cell lysate was subjected to the incubation with antibodies overnight at 4° C. Magnetic protein A/G beads were added to precipitate protein-antibody complex. After four washes in NETN buffer, immunoprecipitated proteins were eluted with Laemmli protein sample buffer. Equal volume of co-IP samples were subjected to 4-12% premade polyacrylamide gels (Invitrogen). Primary antibodies were used for western blot as follows: anti-ETO (Santa Cruz), human and mouse anti-TAF1 (Santa Cruz), anti-AE (Diogenode), anti-AML1 (Cell Signaling Technology), anti-CARM1 (Millipore), anti-CBFβ (Cell Signaling Technology), anti-ID1 (Santa Cruz).

In jurisdictions that forbid the patenting of methods that are practiced on the human body, the meaning of “administering” of a composition to a human subject may be restricted to prescribing a controlled substance that a human subject can self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Results

TAF1 was Required for the Proliferation of AE Expressing Cells:

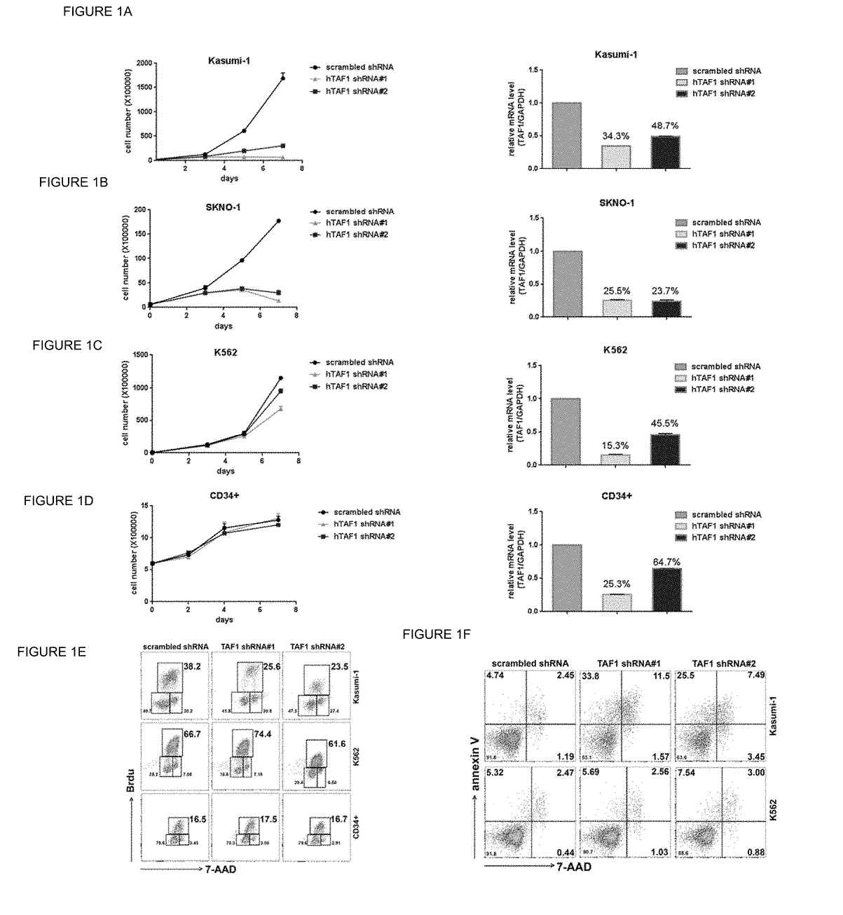

To elucidate the role of TAF1 in the proliferation of AE expressing cells, TAF1 was knocked down (KD) in human Kasumi-1 cells and SKNO-1 cells using two different TAF1 shRNAs that reduce TAF1 expression, compared to cells transduced with a scrambled shRNA (FIGS. 1A and 1B, right panels). KD of TAF1 by either shRNA blocked the proliferation of these cells (FIGS. 1A and 1B, left panels), suggesting that TAF1 was essential for their growth. As a component of the TFIID complex, it is possible that TAF1 is needed for the proliferation of all cells. To evaluate this possibility, TAF1 was knocked down in the non-AE expressing K562 leukemia cell line and in CD34+ hematopoietic stem progenitor cells (HSPCs) isolated from human umbilical cord blood (CB). The reduced expression of TAF1 in these two cell types was comparable to that achieved in Kasumi-1 cells (FIGS. 1C and 1D, right panels); however, decreasing TAF1 expression had little effect on the proliferation of these cells (FIGS. 1C and 1D, left panels). Thus, TAF1 appears to play a particular role in the proliferation of AE expressing cells.

To further assess the effect of TAF1 on cell proliferation, cells were labeled with BrdU and measured the cell cycle profile using flow cytometry. KD of TAF1 reduced the percentage of Kasumi-1 cells in S phase from 38.2 to 25.6 or 23.5, but had no effect on the percentage of K562 cells or CD34+ cells in S phase (FIG. 1E). Thus, it appeared that TAF1 was particularly critical for the proliferation of AE expressing cells.

The Knockdown of TAF1 Induced Apoptosis in AE Expressing Cells:

To determine whether KD of TAF1 triggers apoptosis in AE expressing cells, Kasumi-1 cells and K562 cells were infected with scrambled shRNA or two TAF1 directed shRNAs and stained with Annexin V and 7-AAD. TAF1 KD increased the percentage of apoptotic Kasumi-1 cells without increasing the apoptosis of K562 cells (FIG. 1F). Thus, TAF1 depletion primarily impaired cell cycle progression and induced apoptosis in AE expressing cells.

The Knockdown of TAF1 Promoted Myeloid Differentiation and Blocked the Self-Renewal of Hematopoietic Stem Cells:

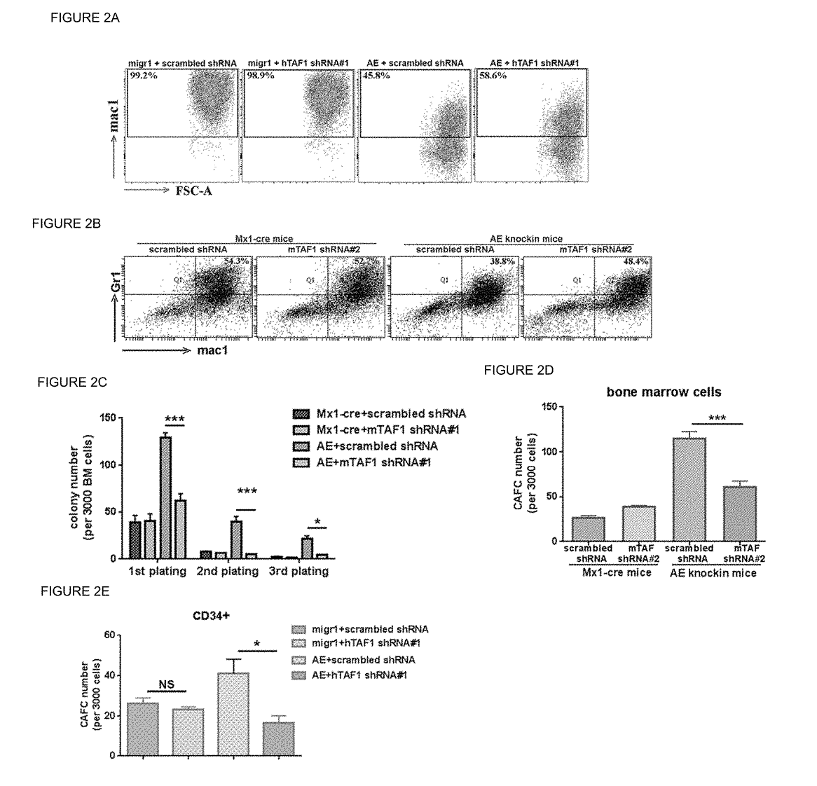

AE blocks the expression of myeloid differentiation markers such as CD11b on human CD34+CB cells 25. To define the role of TAF1 in this effect, TAF1 was knocked down in AE expressing CD34+ cells and in AE conditional knockin mouse bone marrow cells. Increased CD11b (mac1) expression was found on the human cells (FIG. 2A) and increased mac1 and Gr1 staining on the mouse cells compared to Mx1-cre control mice (FIG. 2B). Taken together, it appeared that TAF1 participates critically in the AE mediated block of myeloid differentiation.

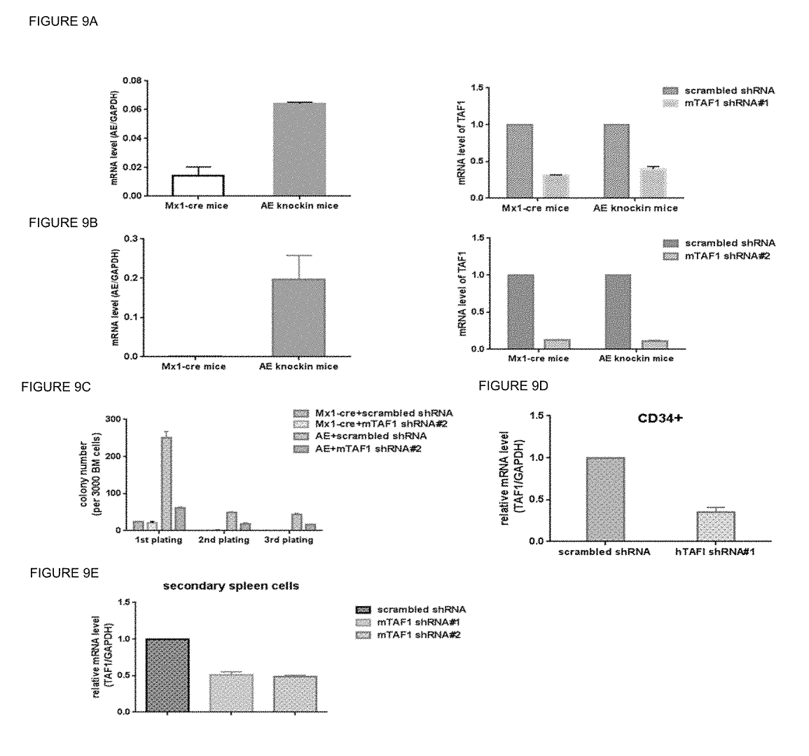

To investigate the role of TAF1 KD in AE driven HSPC self-renewal, bone marrow cells were isolated from AE conditional knockin mice and infected with scrambled shRNA or two different TAF1 directed shRNAs. After confirming AE expression and TAF1 depletion (FIGS. 9A and 9B), serial replating colony formation assays (FIG. 2C and FIG. 9C) and cobblestone area forming (CAFC) assays (FIG. 2D) were performed. TAF1 KD significantly impaired the increased replating capacity and CAFC formation driven by AE. AE expressing CD34+ human CB cells were examined and confirmed that KD of TAF1 (FIG. 9D) impaired cobblestone area formation induced by AE in human as well as mouse HSPCs (FIG. 2E). These data suggested that TAF1 was also involved in AE induced HSPC self-renewal.

The Depletion of TAF1 Blocked Proliferation and Self-Renewal of AE9a Expressing Leukemic Cells:

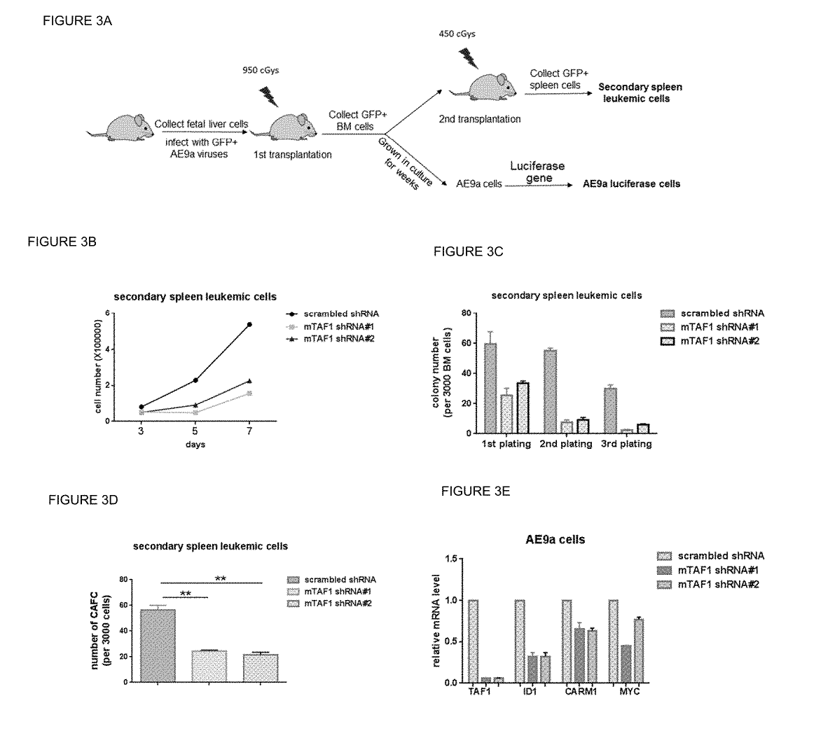

While AE is insufficient to induce leukemia in mice by itself^{26, 27}, expression of the alternatively spliced form of AE, AE exon 9a (AE9a) has been shown to induce leukemia in mice^{28, 29}. To determine whether the lack of TAF1 has same impact on AE9a expressing leukemic cells, AE9a luciferase cells and secondary spleen leukemic cells were developed as shown in FIG. 3A. To determine whether KD of TAF1 affects cell growth of leukemic spleen cells in vitro, the same number of secondary spleen leukemic cells transduced with scrambled or TAF1 directed shRNAs were plated and cell numbers were counted on day 3, 5 and 7. As shown in FIG. 3B and FIG. 9E, depletion of TAF1 blocked the growth of secondary spleen leukemic cells similar to its effects on primary AE expressing cells. To determine the impact of TAF1 depletion on the self-renewal of these leukemic cells, serial replating assays and CAFC assays were performed using secondary spleen leukemic cells infected with scrambled or TAF1 directed shRNAs. As shown in FIG. 3C and FIG. 3D, TAF1 was critical for maintaining the self-renewal and frequency of leukemic stem cells. These data reveal that TAF1 is as indispensable for AE9a expressing leukemic cells as it is for AE expressing cells.

TAF1 Plays a Pivotal Role in AE9a Induced Leukemogenesis:

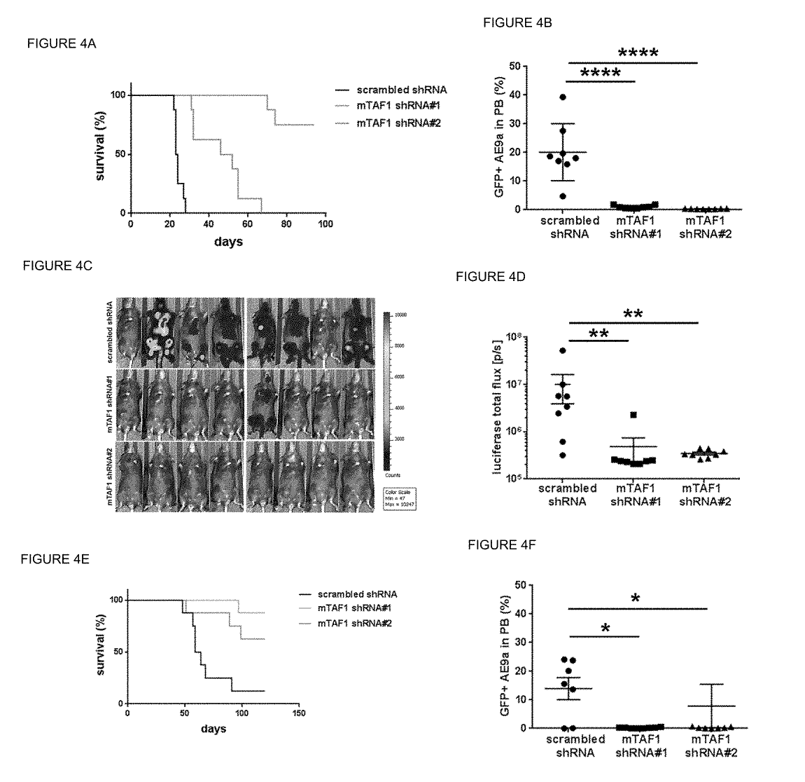

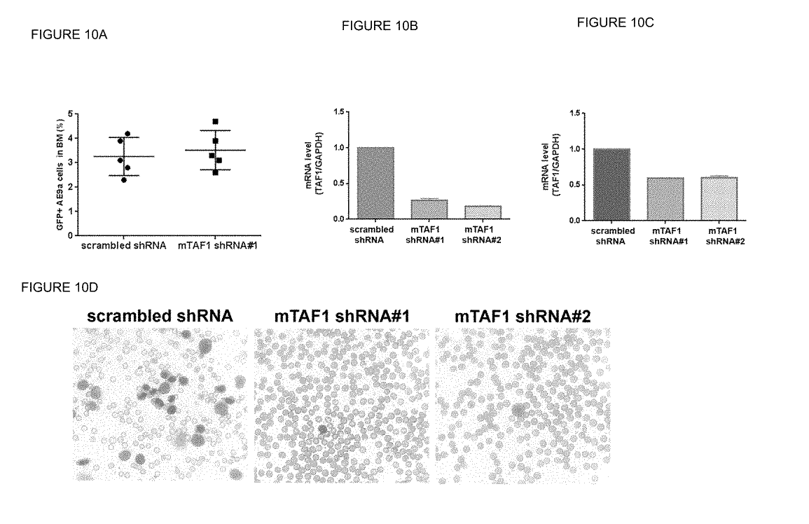

To determine whether TAF1 is involved in AE9a-driven in vivo leukemogenesis, TAF1 was knocked down in AE9a luciferase cells using two mouse TAF1 directed shRNAs. After confirming TAF1 depletion (FIG. 10B), equal numbers of AE9a luciferase cells with normal or KD levels of TAF1 were injected into irradiated mice. TAF1 KD had no influence on the engraftment of the AE9a luciferase cells (FIG. 10A). However, mice injected with TAF1 KD AE9a luciferase cells survived longer than mice injected with AE9a luciferase cells that express wildtype levels of TAF1 (FIG. 4A), implying a pivotal role for TAF1 in leukemia development. Further, the in vivo growth of AE9a leukemic cells in mice was examined using the IVIS imaging system: 20 days after injection of AE9a luciferase cells that also express the luciferase gene, the luciferase signal was widely distributed in the spleen and bone marrow of 6/8 recipient mice. In contrast, only 1/16 mice that received TAF1 depleted AE9a luciferase cells had detectable luciferase signal (FIGS. 4C and 4D). To demonstrate that TAF1 loss impairs the initiation of leukemogenesis, the number of GFP tagged AE9a luciferase cells in the peripheral blood three weeks after injection was quantified using flow cytometry and found that KD of TAF1 significantly diminished their number (FIG. 4B). Secondary spleen leukemic cells were also infected with either scrambled or TAF1 directed shRNAs before injecting them into tertiary recipient mice. After tertiary transplantation, the mice receiving TAF1 KD cells (FIG. 10C) exhibited less GFP+AE9a cells in their peripheral blood (FIG. 4F and FIG. 10D) and had longer survival (FIG. 4E). Together, these data demonstrated that TAF1 contributes critically to AE9a-induced leukemogenesis, and could serve as a potential target for anti-leukemia therapy.

TAF1 is Associated with AE:

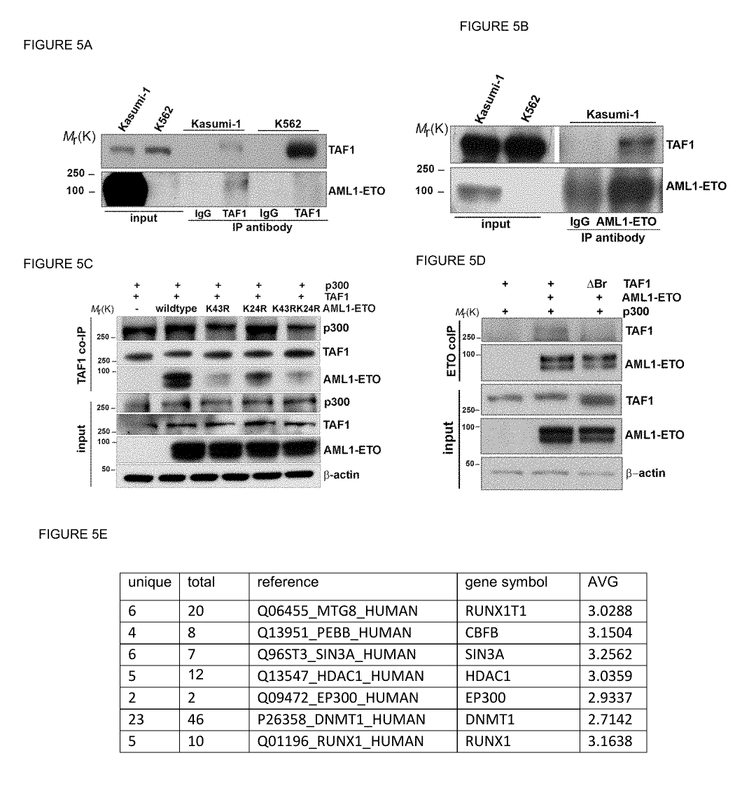

TAF1 was identified based on its binding to an AE peptide in an in vitro peptide pull down assay²⁵. To examine whether the endogenous TAF1 protein associates with full length AE in leukemic cells, reciprocal co-immunoprecipitations using anti-TAF1 and anti-ETO antibodies were performed. As shown in FIGS. 5A and 5B, the physical interaction of TAF1 with AE in Kasumi-1 cells was readily detected, while no association was seen in K562 cells (FIG. 5A). In Kasumi-1 cells, the analysis of Mass Spectrometry following TAF1 immunoprecipitation also confirmed that TAF1 is associated with AML1-ETO (also RUNX1T1) (FIG. 5E). To determine whether acetylation of lysine 43 in AE is important for TAF1 binding 25, full length AE, AE K43R, AE K24R or AE K24RK43R were transfected with TAF1 and p300 into 293T cells and then performed a co-IP using an anti-TAF1 antibody. As shown in FIG. 5C, mutating lysine 43 in AE to arginine impairs the interaction of AE with TAF1, while mutating lysine 24 to arginine had little influence on their association, indicating that lysine 43 acetylation appears to be required for the interaction of AE with TAF1. To determine whether the bromodomains in TAF1 are critical for recognizing K43 acetylation on AE, both bromodomains of TAF1 were deleted, this abrogated the association of TAF1 with AE (FIG. 5D). Thus, it appears that TAF1 binds to acetylated lysine 43 on AE through its bromodomains.

The Expression of AE Target Genes was Affected by the Depletion of TAF1 in Kasumi-1 Cells:

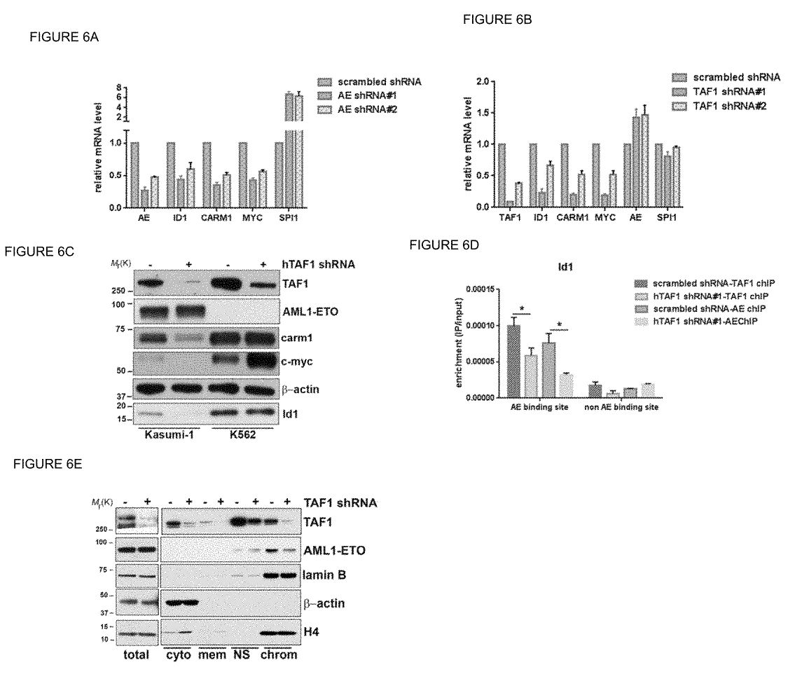

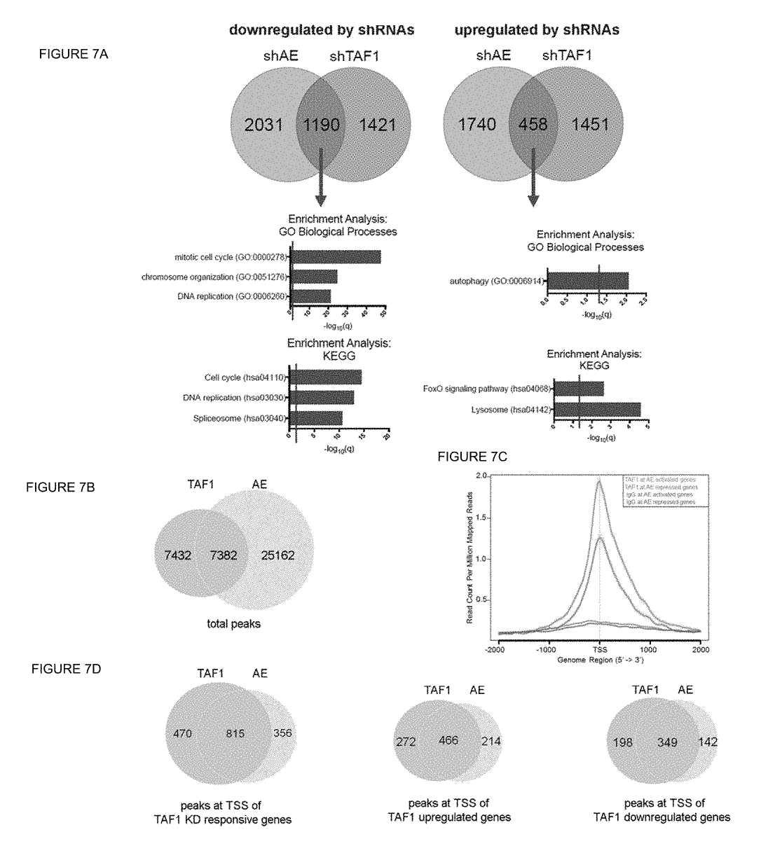

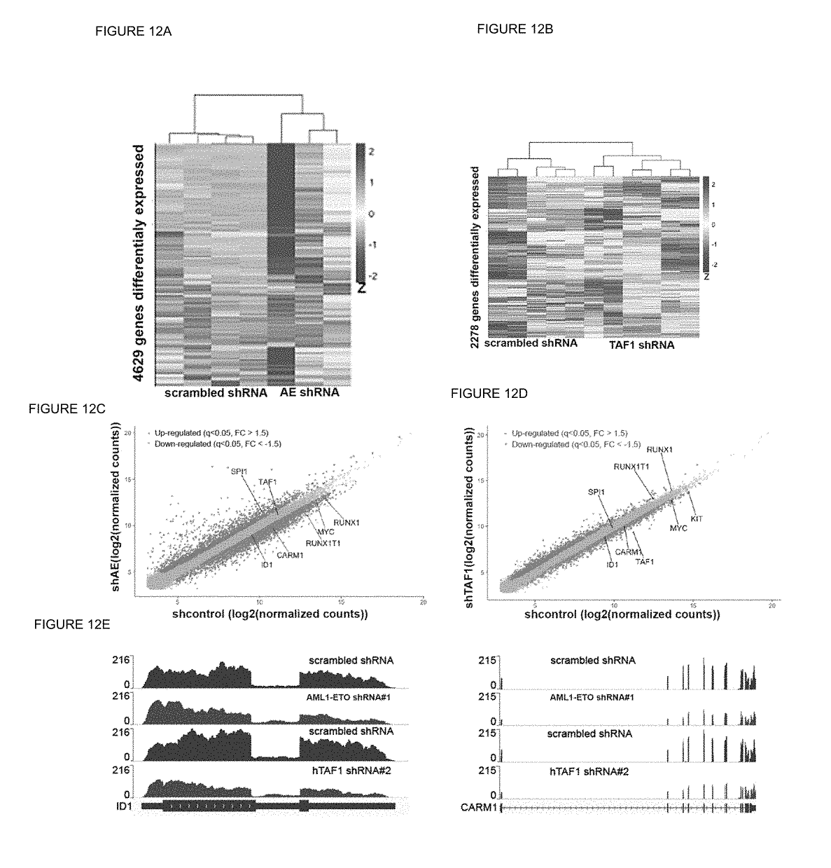

Given the importance of TAF1 in mediating the effects of AE on hematopoietic stem cell biology, how knockdown of TAF1 affects AE regulated gene expression was explored. ID1, CARM1 and MYC are AE activated genes, and confirmed that their expression was reduced by AE KD (FIG. 6A and supplementary FIG. 4C). TAF1 KD also significantly reduced the expression of these genes in Kasumi-1 cells (FIGS. 6B, 6C, FIGS. 12D, and 12E), without reducing the level of AE expression (FIGS. 6B, 6C, and FIG. 12D). Thus, AE works in concert with TAF1 to activate its target genes. In AE9a cells, it was shown that depletion of TAF1 impairs the expression of a subset of AE9a target genes (FIG. 3E). To identify those genes regulated by both TAF1 and AE across the genome, RNA-seq in Kasumi-1 cells was performed (FIGS. 12A and 12B) and found that 37% of AE activated genes are also upregulated by TAF1; GO and KEGG analysis show that these genes control cell cycle (FIG. 7A left panel). Surprisingly, 21% of AE repressed genes are also repressed by TAF1 (FIG. 7B). Clearly, TAF1 is essential for the expression of both AE activated and repressed genes implying that the role of TAF1 in the expression of AE target genes is distinct from its function in PIC.

The Knockdown of TAF1 Reduced the Deposition of AE at its Target Genes:

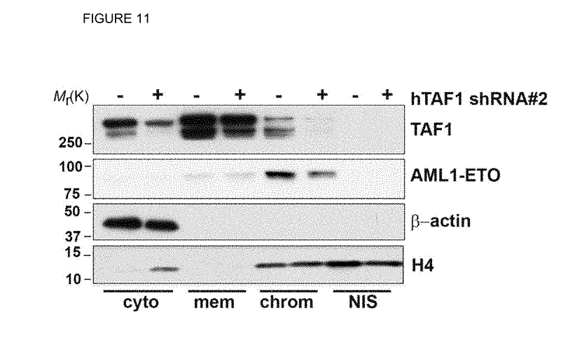

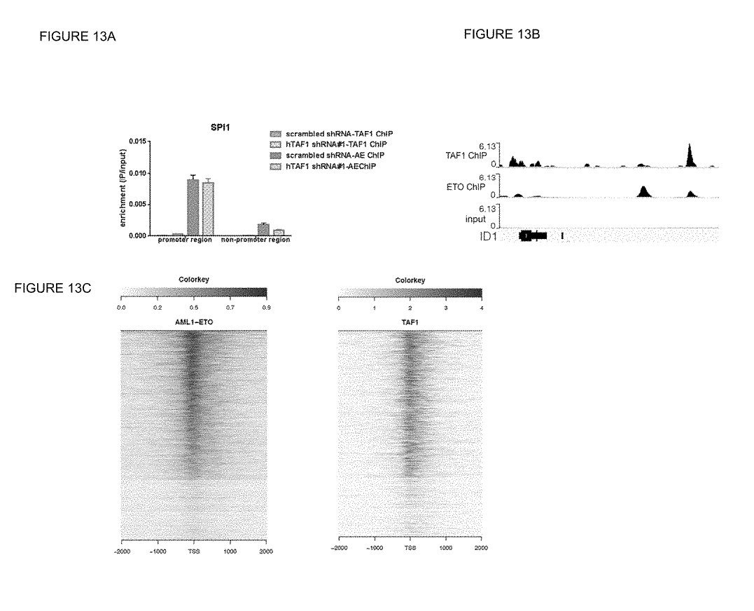

To examine how KD of TAF1 affects the deposition of AE at its target gene regulatory regions, the deposition of TAF1 and AE on ID1 regulatory regions in Kasumi-1 cells that had either wild type or reduced TAF1 levels, was examined using a region where AE does not bind as the negative control for chromatin immunoprecipitation assays. Both TAF1 and AE were found at ID1 regulatory regions, and that KD of TAF1 remarkably decreased the recruitment of AE (FIG. 6D and FIG. 13B). Thus, TAF1 is essential for AE deposition on at least some of its target genes. To further evaluate the deposition of AE on chromatin by TAF1, subcellular fractions of Kasumi-1 cells were generated, as shown in FIG. 6E and supplementary FIG. 11, the KD of TAF1 released AE from the chromatin fraction without changing the overall level of AE in the whole cell lysate. To further define where TAF1 plays an essential role in the recruitment of AE to its target genes, ChIP-seq was performed using an anti-ETO antibody and an anti-TAF1 antibody in Kasumi-1 cells. Analysis of the ChIP-seq data indicated that 50% of the TAF1 peaks co-localize with AE peaks across the genome, while 23% of the AE peaks overlap with TAF1 peaks (FIGS. 7B, 7C, and FIG. 13C). The combined analysis of ChIP-sequencing and RNA-sequencing data demonstrated significant overlap between AE and TAF1 deposition (63% of TAF1 peaks and 70% of AE peaks) at the transcription start site (TSS) of those genes which are differentially expressed (including upregulated and downregulated genes) after TAF1 knockdown (FIG. 7D). Next, the binding of TAF1 and AE at those AE target genes whose expression is not affected by TAF1 KD such as SPI1 (that encodes PU.1) was examined (FIG. 6B and FIG. 12D); ChIP analysis indicated that TAF1 was absent from the SPI1 promoter region while AE was present (FIG. 13A). These data implied that TAF1 facilitated the recruitment of AE to its target genes and coordinately regulated the expression of a subset of AE target genes.

Effect of a TAF1 Bromodomain Inhibitor on the Proliferation of AE Expressing Cells and the Expression of a Subset of AE Regulated Genes:

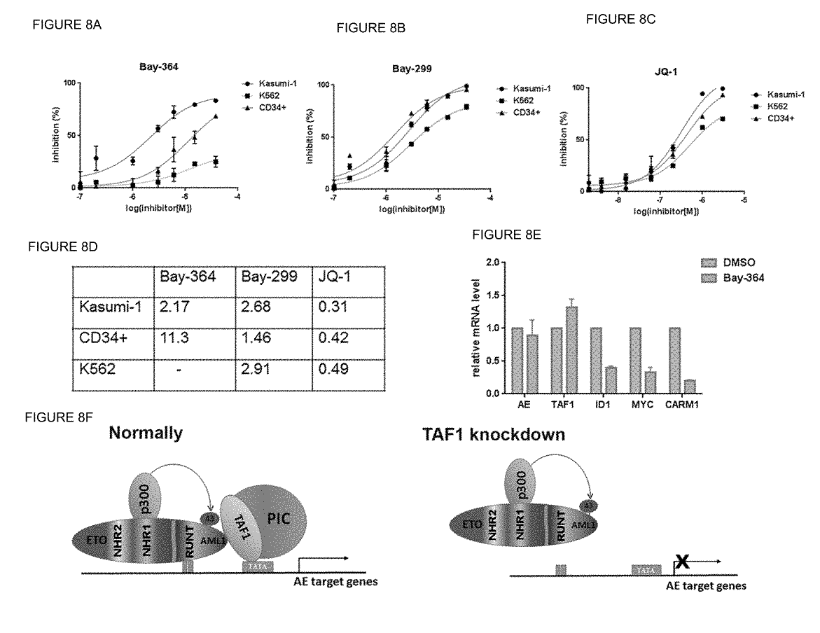

Bay-364 (also Bay-299N) is a commercially available small molecule inhibitor of the second bromodomain in TAF1. Given the essential role of TAF1 in the proliferation of AE expressing cells, the effect of Bay-364 on the growth of Kasumi-1, K562 and CD34+ cells was examined. As shown in FIGS. 8A and 8B, Kasumi-1 cells are more sensitive to Bay-364 treatment than CD34+ cells, while Bay-364 treatment has little influence on the growth of K562 cells. In contrast, Bay-299 which is a bromodomain inhibitor of both BRD1 and TAF1 suppresses the growth of Kasumi-1 cells, but also CD34+ cells and K562 cells (FIGS. 8B and 8D). JQ-1 inhibits the bromodomains of several BET family proteins including BRD2, BRD3, BRD4 and BRDT. Although JQ-1 is more potent than Bay-364 in suppressing the growth of Kasumi-1 cells, the IC50s of JQ-1 on Kasumi-1 cells and CD34+ cells are quite similar, perhaps indicative of its broader effect (FIGS. 8C and 8D). Because Bay-364 selectively suppresses the proliferation of AE expressing cells with much less toxicity on HSPCs, Bay-364 or similarly acting agents, is contemplated as a therapy for AE-expressing leukemia.

To validate that Bay-364 has the same effect as TAF1 KD on AE-mediated gene expression, RNA was extracted from Kasumi-1 cells in presence or absence of Bay-364. ID1, CARM1, MYC genes were chosen as the representatives of AE upregulated genes. Realtime PCR results showed that Bay-364 treatment repressed the expression of these genes as TAF1 KD did (FIG. 8E). Taken together, the TAF1 bromodomain appears to be selectively essential for the survival of AE expressing cells by regulating a critical subset of AE target genes.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

### REFERENCES

1. Andrieu, V. et al. Molecular detection of t(8;21)/AML1-ETO in AML M1/M2: correlation with cytogenetics, morphology and immunophenotype. Br J Haematol 92, 855-865 (1996).
2. Moroi, K. & Sato, T. Comparison between procaine and isocarboxazid metabolism in vitro by a liver microsomal amidase-esterase. Biochem Pharmacol 24, 1517-1521 (1975).
3. Rowley, J. D. Recurring chromosome abnormalities in leukemia and lymphoma. Semin Hematol 27, 122-136 (1990).
4. Erickson, P. et al. Identification of breakpoints in t(8;21) acute myelogenous leukemia and isolation of a fusion transcript, AML1/ETO, with similarity to Drosophila segmentation gene, runt. Blood 80, 1825-1831 (1992).
5. Okuda, T., vanDeursen, J., Hiebert, S. W., Grosveld, G. & Downing, J. R. AML1, the target of multiple chromosomal translocations in human leukemia, is essential for normal fetal liver hematopoiesis. Cell 84, 321-330 (1996).
6. Okuda, T., vanDeursen, J., Hiebert, S. W., Grosveld, G. & Downing, J. R. AML-1, the target of multiple chromosomal translocations in human leukemia, is essential for normal murine fetal liver hematopoiesis. Blood 86, 2372-2372 (1995).
7. Westendorf, J. J. et al. The t(8;21) fusion product, AML-1-ETO, associates with C/EBP-alpha, inhibits C/EBP-alpha-dependent transcription, and blocks granulocytic differentiation. Mol Cell Biol 18, 322-333 (1998).
8. Pabst, T. et al. AML1-ETO downregulates the granulocytic differentiation factor C/EBPalpha in t(8;21) myeloid leukemia. Nat Med 7, 444-451 (2001).
9. Mulloy, J. C. et al. The AML1-ETO fusion protein promotes the expansion of human hematopoietic stem cells. Blood 99, 15-23 (2002).
10. Corsello, S. M. et al. Identification of AML1-ETO modulators by chemical genomics. Blood 113, 6193-6205 (2009).
11. Spirin, P. V. et al. A Comparison of Target Gene Silencing using Synthetically Modified siRNA and shRNA That Express Recombinant Lentiviral Vectors. Acta Naturae 1, 86-90 (2009).
12. Spirin, P. V. et al. Silencing AML1-ETO gene expression leads to simultaneous activation of both pro-apoptotic and proliferation signaling. Leukemia 28, 2222-2228 (2014).
13. Sun, X. J. et al. A stable transcription factor complex nucleated by oligomeric AML1-ETO controls leukaemogenesis. Nature 500, 93-97 (2013).
14. Park, S. et al. Structure of the AML1-ETO eTAFH domain-HEB peptide complex and its contribution to AML1-ETO activity. Blood 113, 3558-3567 (2009).
15. Weinzierl, R. O., Dynlacht, B. D. & Tjian, R. Largest subunit of Drosophila transcription factor IID directs assembly of a complex containing TBP and a coactivator. Nature 362, 511-517 (1993).
16. Maston, G. A. et al. Non-canonical TAF complexes regulate active promoters in human embryonic stem cells. Elife 1, e00068 (2012).
17. D'Alessio, J. A., Wright, K. J. & Tjian, R. Shifting players and paradigms in cell-specific transcription. Molecular cell 36, 924-931 (2009).
18. Sikorski, T. W. & Buratowski, S. The basal initiation machinery: beyond the general transcription factors. Current opinion in cell biology 21, 344-351 (2009).
19. Kedmi, A. et al. Drosophila TRF2 is a preferential core promoter regulator. Genes & development 28, 2163-2174 (2014).
20. O'Brien, T. & Tjian, R. Different functional domains of TAF11250 modulate expression of distinct subsets of mammalian genes. Proc Natl Acad Sci USA 97, 2456-2461 (2000).
21. Suzuki-Yagawa, Y., Guermah, M. & Roeder, R. G. The ts13 mutation in the TAF(II)250 subunit (CCG1) of TFIID directly affects transcription of D-type cyclin genes in cells arrested in G1 at the nonpermissive temperature. Mol Cell Biol 17, 3284-3294 (1997).
22. Oh, H. R., An, C. H., Yoo, N. J. & Lee, S. H. Frameshift Mutations in the Mononucleotide Repeats of TAF1 and TAF1L Genes in Gastric and Colorectal Cancers with Regional Heterogeneity. Pathol Oncol Res (2016).
23. Ribeiro, J. R., Lovasco, L. A., Vanderhyden, B. C. & Freiman, R. N. Targeting TBP-Associated Factors in Ovarian Cancer. Front Oncol 4, 45 (2014).
24. Tavassoli, P. et al. TAF1 differentially enhances androgen receptor transcriptional activity via its N-terminal kinase and ubiquitin-activating and -conjugating domains. Mol Endocrinol 24, 696-708 (2010).
25. Wang, L. et al. The leukemogenicity of AML1-ETO is dependent on site-specific lysine acetylation. Science 333, 765-769 (2011).
26. Licht, J. D. AML1 and the AML1-ETO fusion protein in the pathogenesis of t(8;21) AML. Oncogene 20, 5660-5679 (2001).
27. Higuchi, M. et al. Expression of a conditional AML1-ETO oncogene bypasses embryonic lethality and establishes a murine model of human t(8;21) acute myeloid leukemia. Cancer Cell 1, 63-74 (2002).
28. Yan, M. et al. A previously unidentified alternatively spliced isoform of t(8;21) transcript promotes leukemogenesis. Nature medicine 12, 945-949 (2006).
29. Hatlen, M. A. et al. Integrative genetic analysis of mouse and human AML identifies cooperating disease alleles. J Exp Med 213, 25-34 (2016).
30. Roeder, R. G. The role of general initiation factors in transcription by RNA polymerase II. Trends Biochem Sci 21, 327-335 (1996).
31. Goodrich, J. A., Cutler, G. & Tjian, R. Contacts in context: promoter specificity and macromolecular interactions in transcription. Cell 84, 825-830 (1996).
32. Thomas, M. C. & Chiang, C. M. The general transcription machinery and general cofactors. Crit Rev Biochem Mol Biol 41, 105-178 (2006).
33. Thut, C. J., Chen, J. L., Klemm, R. & Tjian, R. p53 transcriptional activation mediated by coactivators TAFII40 and TAFII60. Science 267, 100-104 (1995).
34. Kadonaga, J. T. The DPE, a core promoter element for transcription by RNA polymerase II. Exp Mol Med 34, 259-264 (2002).
35. Wright, K. J., Marr, M. T., 2nd & Tjian, R. TAF4 nucleates a core subcomplex of TFIID and mediates activated transcription from a TATA-less promoter. Proc Natl Acad Sci USA 103, 12347-12352 (2006).
36. Louder, R. K. et al. Structure of promoter-bound TFIID and model of human pre-initiation complex assembly. Nature 531, 604-609 (2016).
37. Bieniossek, C. et al. The architecture of human general transcription factor TFIID core complex. Nature 493, 699-702 (2013).
38. Mandoli, A. et al. CBFB-MYH11/RUNX1 together with a compendium of hematopoietic regulators, chromatin modifiers and basal transcription factors occupies self-renewal genes in inv(16) acute myeloid leukemia. Leukemia 28, 770-778 (2014).
39. Shirakawa, K. et al. Salicylate, diflunisal and their metabolites inhibit CBP/p300 and exhibit anticancer activity. Elife 5 (2016).
40. Wang, L. et al. Regulation of AKT signaling by Id1 controls t(8;21) leukemia initiation and progression. Blood 126, 640-650 (2015).
41. Tzelepis, K. et al. A CRISPR Dropout Screen Identifies Genetic Vulnerabilities and Therapeutic Targets in Acute Myeloid Leukemia. Cell Rep 17, 1193-1205 (2016).

1. A method of treating leukemia, the method comprising administering to mammalian subject in need thereof an inhibitor of Transcription initiation factor TFIID subunit 1 (TAF1).

2. (canceled)

3. The method of claim 1, wherein the subject is a human.

4. The method of claim 1, wherein the leukemia is Acute Myelogenous Leukemia (AML).

5. The method of claim 1, wherein the leukemia is Acute Myelogenous Leukemia 1-Eight Twenty One oncoprotein (AML1-ETO) expressing leukemia.

6. The method of claim 1, wherein the inhibitor of TAF1 is a TAF1 bromodomain inhibitor.

7. The method of claim 6, wherein the TAF1 bromodomain inhibitor is selected from the group consisting of:
(a) Bay-364 (6-(3-Hydroxy-propyl)-2-(1-methyl-2-oxo-2,3-dihydro-1H-benzoimidazol-5-yl)-benzo[de]isoquinoline);
(b) Bay-299 (6-(3-Hydroxypropyl)-2-(1,3,6-trimethyl-2-oxo-2,3-dihydro-1Hbenzimidazol-5-yl)-1H-benzo[de]isoquinoline-1,3(2H)-dione),
(c) 2-(1,3,6-Trimethyl-2-oxo-2,3-dihydro-1H-benzimidazol-5-yl) 1Hbenzo[de]isoquinoline-1,3(2H)-dione;
(d) 2-[6-(Dimethylamino)-1,3-dimethyl-2-oxo-2,3-dihydro-1H-benzimidazol-5-yl]-1H-benzo[de]isoquinoline-1 ,3(2H)-dione;
(e) 2-(6-Bromo-1,3-dimethyl-2-oxo-2,3-dihydro-1H-benzimidazol-5-yl)-1H-benzo[de]isoquinoline-1 ,3(2H)-dione;
(f) 6-Bromo-2-(1,3,6-trimethyl-2-oxo-2,3-dihydro-1H-benzimidazol5-yl)-1H-benzo[de]isoquinoline-1 ,3(2H)-dione;
(g) 6-Chloro-2-(1,3,6-trimethyl-2-oxo-2,3-dihydro-1H-benzimidazol5-yl)-1H-benzo[de]isoquinoline-1,3(2H)-dione;
(h) 5-Nitro-2-(1,3,6-trimethyl-2-oxo-2,3-dihydro-1H-benzimidazol-5-yl)-1H-benzo[de]isoquinoline-1,3(2H)-dione;
(i) 5-Amino-2-(1,3,6-trimethyl-2-oxo-2,3-dihydro-1H-benzimidazol5-yl)-1H-benzo[de]isoquinolone-1,3(2H)-dione;
(j) 5-Hydroxy-2-(1,3,6-trimethyl-2-oxo-2,3-dihydro-1H-benzimidazol-5-yl)-1H-benzo[de]isoquinoline-1,3(2H)-dione;
(k) 5-Bromo-2-(1,3,6-trimethyl-2-oxo-2,3-dihydro-1H-benzimidazol5-yl)-1H-benzo[de]isoquinoline-1,3(2H)-dione; and
(l) 1,3-Dioxo-2-(1,3,6-trimethyl-2-oxo-2,3-dihydro-1H-benzimidazol-5-yl)-2,3-dihydro-1H-benzo[de]isoquinoline-5-carbonitrile.

8. A method of reducing the risk of leukemia, the method comprising administering to mammalian subject in need thereof an inhibitor of Transcription initiation factor TFIID subunit 1 (TAF1).

9. The method of claim 8, wherein the subject is a human.

10. The method of claim 8, wherein the leukemia is Acute Myelogenous Leukemia (AML).

11. The method of claim 8, wherein the leukemia is Acute Myelogenous Leukemia 1-Eight Twenty One oncoprotein (AML1-ETO) expressing leukemia.

12. The method of claim 8, wherein the inhibitor of TAF1 is a TAF1 bromodomain inhibitor.

13. The method of claim 12, wherein the TAF1 bromodomain inhibitor is selected from the group consisting of:
(a) Bay-364 (6-(3-Hydroxy-propyl)-2-(1-methyl-2-oxo-2,3-dihydro-1H-benzoimidazol-5-yl)-benzo[de]isoquinoline);
(b) Bay-299 (6-(3-Hydroxypropyl)-2-(1,3,6-trimethyl-2-oxo-2,3-dihydro-1Hbenzimidazol-5-yl)-1H-benzo[de]isoquinoline-1,3(2H)-dione),
(c) 2-(1,3,6-Trimethyl-2-oxo-2,3-dihydro-1H-benzimidazol-5-yl) 1Hbenzo[de]isoquinoline-1,3(2H)-dione;
(d) 2-[6-(Dimethylamino)-1,3-dimethyl-2-oxo-2,3-dihydro-1H-benzimidazol-5-yl]-1H-benzo[de]isoquinoline-1,3(2H)-dione;
(e) 2-(6-Bromo-1,3-dimethyl-2-oxo-2,3-dihydro-1H-benzimidazol-5-yl)-1H-benzo[de]isoquinoline-1,3(2H)-dione;
(f) 6-Bromo-2-(1,3,6-trimethyl-2-oxo-2,3-dihydro-1H-benzimidazol5-yl)-1H-benzo[de]isoquinoline-1,3(2H)-dione;
(g) 6-Chloro-2-(1,3,6-trimethyl-2-oxo-2,3-dihydro-1H-benzimidazol5-yl)-1H-benzo[de]isoquinoline-1,3(2H)-dione;
(h) 5-Nitro-2-(1,3,6-trimethyl-2-oxo-2,3-dihydro-1H-benzimidazol-5-yl)-1H-benzo[de]isoquinoline-1,3(2H)-dione;
(i) 5-Amino-2-(1,3,6-trimethyl-2-oxo-2,3-dihydro-1H-benzimidazol5-yl)-1H-benzo[de]isoquinolone-1,3(2H)-dione;
(j) 5-Hydroxy-2-(1,3,6-trimethyl-2-oxo-2,3-dihydro-1H-benzimidazol-5-yl)-1H-benzo[de]isoquinoline-1 ,3(2H)-dione;
(k) 5-Bromo-2-(1,3,6-trimethyl-2-oxo-2,3-dihydro-1H-benzimidazol5-yl)-1H-benzo[de]isoquinoline-1 ,3(2H)-dione; and
(l) 1,3-Dioxo-2-(1,3,6-trimethyl-2-oxo-2,3-dihydro-1H-benzimidazol-5-yl)-2,3-dihydro-1H-benzo[de]isoquinoline-5-carbonitrile.
